(19) United States
(12) Patent Application Publication
(10) Pub. No.: US 2012/0115805 A1
(43) Pub. Date: May 10, 2012

(54) USE OF AT LEAST ONE NUCLEIC ACID TO INFLUENCE THE NATURAL PIGMENTATION PROCESS

(75) Inventors: Melanie Giesen, Geldern (DE); Andreas Bock, Dusseldorf (DE); Erik Schulze zur Wiesche, Hamburg (DE)

(73) Assignee: Henkel AG & Co. KGaA, Dusseldorf (DE)

(21) Appl. No.: 13/354,565

(22) Filed: Jan. 20, 2012

Related U.S. Application Data
(63) Continuation of application No. PCT/EP2010/060025, filed on Jul. 13, 2010.

(30) Foreign Application Priority Data
Jul. 23, 2009 (DE) 10 2009 027 955.5
Sep. 24, 2009 (DE) 10 2009 044 972.8

(51) Int. Cl. A61K 31/708; A61P 17/00

(52) U.S. Cl. 514/48; 514/45

(57) ABSTRACT

A method for influencing the natural pigmentation process of skin or appendages thereof, includes the step of topically contacting the skin or appendages thereof with at least one nucleic acid containing at least 55% guanine nucleotide.

### CROSS-REFERENCES TO RELATED APPLICATIONS

This application is a continuation of PCT/EP2010/060025, filed on Jul. 13, 2010, which claims priority under 35 U.S.C. §119 to DE 10 2009 027 955.5 filed on Jul. 23, 2009, and DE 10 2009 044 972.8 filed on Sep. 24, 2009, all of which are hereby incorporated by reference.

### FIELD OF THE INVENTION

The present invention generally relates to the use of nucleic acid(s) in order to influence the natural pigmentation process of skin and/or skin appendages.

### BACKGROUND OF THE INVENTION

In addition to its intrinsic physiological function, such as heat insulation and light protection, hair has a psychosocial function that must not be underestimated. Among other things it provides a means of interpersonal communication and represents a symbol of one's individuality. Changes, such as graying for example, can be enormously damaging to the self-confidence of the person concerned.

Hitherto, rather than combating the causes of hair graying, the hair has been treated with chemical colors that are often aggressive and hence damaging to the hair in order to cover the gray. Moreover, customers frequently complain of a lack of tolerance (itching, burning, prickling) and sustainability (affected areas have to be recolored at regular intervals). The effectiveness of the few biological products currently available on the market has not been scientifically proven and is often doubtful. Significantly effective, biologically active ingredients that influence the graying process directly at the root are not in use.

Pigmentation in the hair follicle is controlled by a defined and complex set of molecular signals. As melanogenesis in grayed follicles is evidently influenced, it can be assumed that the function of parts of this network is modified in the grayed follicle. One consequence of this is the reduction of melanin synthesis, which leads to graying of the follicle. The complex set of molecular signals that influence melanogenesis include inter alia the expression of MCR1 (melanocortin receptor 1), gp100 and ckit. MCR1 and ckit are receptors that transmit key signals of melanogenesis into the cell's interior by binding their ligands alpha-melanocyte stimulating hormone and stem cell factor. Gp100 is a protein of the melanosomal membrane and also regulates other proteins of relevance to melanogenesis. As these parameters are of essential significance in hair follicle pigmentation, it is advantageous to influence these parameters if melanin synthesis in the hair follicle cells is to be maintained or reactivated by the application of a test formulation. Retaining the pigmentation and hence the youthfulness of the hair by means of appropriate active ingredient formulations is a challenge for cosmetic research.

Accordingly, it is desirable to provide active ingredients or combinations of active ingredients and agents containing them that are suitable for influencing the natural pigmentation process, in particular in the hair or hair follicle, without exhibiting the aforementioned disadvantages of the methods known in the prior art for influencing hair color or the degree of hair graying and the youthful appearance of the hair.

Furthermore, other desirable features and characteristics of the present invention will become apparent from the subsequent detailed description of the invention and the appended claims, taken in conjunction with the accompanying drawings and this background of the invention.

### BRIEF SUMMARY OF THE INVENTION

The above needs and others are met by a method for influencing the natural pigmentation process of skin and/or skin appendages, in particular for stimulating the natural pigmentation process, in particular melanogenesis and/or pigmentation of the hair, for preventing and/or reducing graying of the hair and/or for repigmenting gray hair. The method includes topically contacting the hair and/or skin with at least one nucleic acid containing at least 55% guanine nucleotide. The above needs and others are further met by a hair treatment agent containing at least one nucleic acid containing at least 55% guanine nucleotide, 0.1 to 90 wt. % of at least one monohydric alcohol selected from ethanol, n-propanol, isopropanol, and n-butanol, and 0 to 10 wt. % of at least one gelling agent.

### DETAILED DESCRIPTION OF THE INVENTION

The following detailed description of the invention is merely exemplary in nature and is not intended to limit the invention or the application and uses of the invention. Furthermore, there is no intention to be bound by any theory presented in the preceding background of the invention or the following detailed description of the invention.

The present invention involves methods in which at least one nucleic acid containing at least 55% guanine nucleotide is used in order to influence the natural pigmentation process of skin and/or skin appendages.

The present invention also involves methods in which at least one nucleic acid containing at least 55% guanine nucleotide is used in order to influence the natural pigmentation process of skin and/or skin appendages.

Within the meaning of the present invention the term “influencing the natural pigmentation process” is understood to mean the positive or negative influencing of the natural coloring/coloration and/or pigmentation of the skin and/or skin appendages, in particular the stimulation or the partial or complete inhibition of the natural, i.e. biological, pigmentation process in the skin and/or skin appendages, in particular hair or hair follicles.

Within the context according to the invention, skin and skin appendages are understood to be the skin, mucous membranes, hair and hair follicles, glands and nails, in particular the skin, mucous membranes, hair and hair follicles. The term skin is particularly preferably understood to be the skin excluding the mucous membranes. The term skin appendages is most particularly preferably understood to be the hair and hair follicles, preferably body hair, beard hair and head hair, most particularly preferably beard hair and head hair, most particularly preferably head hair and the corresponding hair follicles.

According to a preferred embodiment, the influencing of the natural pigmentation process is understood to be the positive or negative influencing of at least one sub-step of the natural pigmentation process. This influencing relates in particular to the regulation of the molecular signals that influence the biological or natural pigmentation process.

The regulation of the biological or natural pigmentation process through gene regulation, i.e. regulation at an expression level, and/or enzyme regulation, i.e. regulation at an activity level, and/or regulation at a hormone level is preferred.

The regulation of melanogenesis, inter alia regulation of the gene expression of MCR1 (melanocortin receptor 1), gp100 and ckit, is particularly preferred. The regulation of tyrosinase, both of the gene expression of tyrosinase and regulation at an enzyme level, is moreover also encompassed.

According to a preferred embodiment the natural pigmentation process of the hair is influenced, in particular stimulated or prompted. In particular, influencing is understood to be the positive influencing, preferably the positive regulation (up-regulation or activation or prompting or increase) that leads to a stimulation of the natural, biological pigmentation process. Stimulation of melanogenesis in the human hair follicle, in particular of the head hair (the hair follicle located on the scalp/top of the head), is particularly preferred.

According to the invention the pigmentation process, in particular melanogenesis, of the skin and skin appendages, preferably of the hair or hair follicle, can be influenced. In particular, the natural pigmentation process, in particular melanogenesis, in mammals, particularly preferably in humans, can be influenced. The pigmentation process, preferably melanogenesis, of the human hair or human hair follicle, is preferably influenced.

According to the invention stimulation of melanogenesis, preferably melanogenesis in the hair follicle, is particularly preferably understood to be the stimulation, increase, prompting or improvement of melanin synthesis in the melanocytes (preferably the melanocytes in the hair follicle). This is achieved for example by an increase in the gene expression of signal molecules such as MCR1 (melanocortin receptor 1), gp100 and ckit. According to a preferred embodiment, the influencing, preferably stimulation, of melanogenesis is achieved by the use according to the invention. In particular, melanogenesis is stimulated in the hair or hair follicle of the haired scalp and/or beard, in particular in humans.

Within the meaning of the present invention, stimulation of pigmentation is understood in particular to be the improvement, increase and/or stimulation of the transport of melanosomes into the keratinocytes surrounding the hair follicle and also the pigmentation of the individual hair, a selection of hairs, in particular an area of haired skin, in particular scalp, or of the entire head and/or beard hair, that is perceptible to the eye or by correspondingly suitable measuring methods.

Surprisingly it has been found that the use according to the invention of at least one nucleic acid containing at least 55% guanine nucleotide is suitable for stimulating or improving the pigmentation of the hair.

In the context of a preferred embodiment, hair graying, in particular of human hair, is prevented, preferably substantially prevented, and/or reduced by the use according to the invention. Within the meaning of the present invention, hair graying is understood to mean both the visually perceptible graying of hair due to the mixing of white and pigmented hair and the pigment dilution in an individual hair, in other words the graying of an individual hair.

A prevention of hair graying occurs in particular in hair that is not yet grayed, whereas a reduction of hair graying can take place both in already grayed hair and in hair that is not yet grayed. In the one case, hair follicles in which melanogenesis does not function or no longer functions or does not function completely or is disrupted or reduced are prompted/stimulated to melanogenesis again, whereas in hair/hair follicles that are not grayed, a disruption, reduction or down-regulation of melanogenesis does not occur at all or occurs only to a lesser extent.

According to a further preferred embodiment, hair that is already grayed is repigmented by the use according to the invention of at least one nucleic acid containing at least 55% guanine nucleotide.

According to a further particularly preferred embodiment the use according to the invention is a cosmetic use that is non-therapeutic.

In particular, the use according to the invention, which is aimed at hair graying, in particular non-pathological gradual hair graying, arising from the natural aging process, is a purely cosmetic use that does not constitute treatment and/or prevention of a disease and hence is non-therapeutic.

According to a particular embodiment, the use according to the invention takes place topically, i.e. by application onto the skin and/or skin appendages, in particular facial skin and/or the scalp, in particular the scalp.

According to a further preferred embodiment, hair that is already grayed is repigmented by the use according to the invention of at least one nucleic acid containing at least 55% guanine nucleotide.

Surprisingly it has been found that a use of at least one nucleic acid containing at least 55% guanine nucleotide is capable of positively influencing, in particular stimulating, the natural pigmentation process, in particular in the hair or hair follicle, in a synergistic manner. The combination according to the invention induced both the gene expression of MCR-1 and that of ckit and gp100 in a synergistic manner Furthermore, a synergistic increase in melanin synthesis was able to be observed.

The natural pigmentation process of skin and/or skin appendages can thus be influenced, in particular stimulated, by the application of the combination according to the invention or of agents used according to the invention. In particular, the natural pigmentation process of the hair or hair follicle or in the hair follicle can thus be influenced, in particular stimulated. The agents used according to the invention are suitable for stimulating and/or improving the pigmentation of the hair, stimulating melanogenesis, in particular in the hair follicle, preventing and/or reducing hair graying and repigmenting gray hair.

Nucleic acids within the meaning of the present invention are molecules consisting of a plurality of nucleotides. A nucleotide is constructed from three constituents: a phosphoric acid (monophosphate), a monosaccharide having 5 C atoms, also known as pentose, which is present as a five-membered ring (furanose ring), and one of the five nucleobases, namely adenine (A), guanine (G), cytosine (C), thymine (T) or uracil (U).

The term nucleic acid is understood according to the invention to mean single-stranded or double-stranded nucleic acid, which can be of natural or synthetic origin. It can also be in hydrolyzed, partially hydrolyzed or denatured form. The nucleic acid is preferably selected from synthetic nucleic acids, nucleic acids of eukaryotic origin, such as nucleic acids from fish roe or wheat germ, and nucleic acids of bacterial origin, in particular from nucleic acids from Escherichia coli and Clostridium perfringens. Nucleic acids selected from DNA and RNA, in particular low-molecular-weight bacterial DNA, low-molecular-weight eukaryotic DNA, are preferably used according to the invention. Nucleic acids from a single-stranded DNA are most particularly preferred. Mixtures of two or more nucleic acids can also be used according to the invention. According to the invention the nucleic acid contains at least 55% guanine nucleotide in the nucleic acid sequence. The percentages relate to the frequency of the number of guanine nucleotides relative to the total number of nucleotides in the complete nucleic acid. According to a preferred embodiment the nucleic acid has in particular 65%, preferably at least 75%, in particular preferably at least 80%, particularly preferably at least 85%, most particularly preferably at least 90%, extremely preferably at least 95% guanine nucleotide in the nucleic acid sequence. Nucleic acids that can be used according to the invention have a chain length from 4 to 100, in particular 7 to 50, preferably 8 to 30, preferably 10 to 25 and most particularly preferably 12 to 22 nucleotides. According to a preferred embodiment the at least one nucleic acid has a succession of at least 4, preferably at least 5, in particular preferably at least 6, extremely preferably at least 7 or more guanine nucleotides. This means that the at least one nucleic acid that is suitable according to the invention contains a plurality of guanine (G) nucleotides in succession. Within the context according to the invention a succession in a nucleic acid sequence is preferably understood to mean that the nucleic acid sequence that is suitable according to the invention contains multiple, at least 2 (≧2), preferably at least 3 (≧3), in particular at least 4 (≧4), most particularly preferably at least 5 or more (≧5), in particular preferably 6 (≧6), extremely preferably at least 7 (≧7), identical nucleotides in succession.

According to a most particularly preferred embodiment the nucleic acid is a G homopolymer, i.e. the nucleic acid contains only guanine nucleotides. The content of guanine nucleotides in the nucleic acid sequence is correspondingly 100%. G homopolymers having a chain length from 8 to 30, in particular 10 to 25, most particularly preferably 12 to 20 guanine nucleotides are particularly preferred. Most particularly preferred nucleotides are the following nucleotides having the aforementioned preferred percentages by weight in an agent used according to the invention: at least one 12 G homopolymer, at least one 13 G homopolymer, at least one 14 G homopolymer, at least one 15 G homopolymer, at least one 16 G homopolymer, at least one 17 G homopolymer, at least one 18 G homopolymer, at least one 19 G homopolymer, at least one 20 G homopolymer, at least one 21 G homopolymer and at least one 22 G homopolymer. These homopolymers can be linked by phosphodiester bonds and/or phosphorothioate bonds.

A mixture of two or more nucleic acids that is preferably to be used according to the invention must contain at least one nucleic acid that is preferred according to the invention (as described above, the G-rich nucleic acids (with a G content of at least 65% or higher)). The other nucleic acids can differ from the nucleic acids that are preferred according to the invention. Mixtures containing two, three and more of the particularly preferred G-rich nucleic acids (with a G content of at least 65% or higher (see above)) and/or in particular at least one G homopolymer can preferably also be used.

The nucleobases of the at least one nucleic acid used according to the invention can be methylated or non-methylated.

The nucleic acids that can be used according to the invention can be completely (all nucleotides) or partially (only some nucleotides) chemically modified in the manner known to the person skilled in the art. Preferred modifications are, for example:
a) Modification of the internucleoside bridges: exchange of phosphodiesters for methyl phosphonates, phosphoramidates, phosphorothioates (PTO) or hydroxylamines;
b) Modification of the sugar components: exchange of ribose for various hexo- or pentopyranoses or 3′-5′-carbocyclically bridged derivatives of 2′-deoxyribose (Steffens R & Leumann C J: Tricyclo-DNA: A phosphodiester-backbone based DNA analog exhibiting strong complementary base-pairing properties. J Am Chem Soc; 119: 11548-11549, 1997);
c) Exchange of the strand backbone: exchange of the polyester chains based on sugar phosphate units for carboxamide chains based on amino acid derivatives such as N-(2-aminoethyl)glycine units.

According to a preferred embodiment the nucleic acid according to the invention preferably has at least one, preferably 2 and more, or entirely phosphorothioate linkages. Phosphodiesters, phosphorothioate-phosphodiester mixmers or phosphorothioates are particularly preferred according to the invention.

According to a preferred embodiment the at least one nucleic acid containing at least 55% guanine nucleotide is used in a cosmetic agent that contains the at least one nucleic acid containing at least 55% guanine nucleotide in a total amount from 0.0000001 to 5 wt. %, preferably 0.000001 to 1 wt. %, particularly preferably 0.00001 to 0.1 wt. %, exceptionally preferably 0.00005 to 0.1 wt. %, relative in each case to the total weight of the agent.

Over and above the influencing of the natural pigmentation, the cosmetic agents used according to the invention exhibit improved care effects on skin and hair.

The positive effects are clearly pronounced on keratinic fibers in particular, such that preferred cosmetic agents according to the invention are hair treatment agents.

Hair treatment agents within the meaning of the present invention are for example hair coloring agents, bleaching agents, hair shampoos, hair conditioners, conditioning shampoos, hair sprays, hair rinses, hair masks, hair packs, hair tonics, permanent wave fixing solutions, hair coloring shampoos, hair coloring agents, hair fixing agents, hair setting agents, hair styling preparations, blow-drying lotions, styling mousses, hair gels, hair waxes or combinations thereof Particularly preferred hair treatment agents have the characterizing feature that they are formulated as a shampoo, hair tonic, hair mask, hair rinse, hair mousse, hair fixing agent, hair spray, hair gel and/or hair coloring agent. These agents are particularly advantageous in view of the fact that for reasons of time and convenience the consumer often shies away from the use of multiple different agents and/or multiple application steps.

According to a preferred embodiment at least one hair conditioning agent selected from cationic polymers, cationic surfactants, silicones and/or vegetable oils is preferably additionally included.

The agents for use according to the invention can contain further active ingredients and auxiliary substances. These are described below.

The agents used according to the invention preferably additionally contain at least one emulsifier or surfactant. The compositions for use according to the invention can contain surfactants, in particular cationic surfactants. Protection is or can be requested for surfactant-containing agents for use according to the invention; surfactants, in particular cationic surfactants, contribute to the technical aim of the invention and hence to the solution of the technical object underlying the invention according to the application. Preferred surfactants and the amounts in which they are contained in the compositions according to the invention are disclosed in the priority document DE 102009044972 on pages 8 to 18; the features cited therein clearly belong implicitly to the description of the invention contained in the submitted application and hence to the disclosure of said application.

Particularly preferred hair treatment agents according to the invention have the characterizing feature that as a cationic care substance they contain, relative to their weight, 0.05 to 7.5 wt. %, preferably 0.1 to 5 wt. %, particularly preferably 0.2 to 3.5 wt. % and in particular 0.25 to 2.5 wt. % of cationic surfactant(s) from the group of quaternary ammonium compounds and/or esterquats and/or amido amines, wherein preferred cationic surfactant(s) is/are selected from alkyl trimethylammonium chlorides having preferably 10 to 18 carbon atoms in the alkyl residue and/or diallyl dimethylammonium chlorides having preferably 10 to 18 carbon atoms in the alkyl residue and/or trialkyl methylammonium chlorides having preferably 10 to 18 carbon atoms in the alkyl residue and/or cetyl trimethylammonium chloride and/or stearyl trimethylammonium chloride and/or distearyl dimethylammonium chloride and/or lauryl dimethylammonium chloride and/or lauryl dimethyl benzylammonium chloride and/or tricetyl methylammonium chloride and/or Quatemium-27 and/or Quatemium-83 and/or N-methyl-N(2-hydroxyethyl)-N,N-(ditalgacyloxyethyl)ammonium methosulfate and/or N-methyl-N(2-hydroxyethyl)-N,N-(distearoyloxyethyl)ammonium methosulfate and/or N,N-dimethyl-N,N-distearoyloxyethyl ammonium chloride and/or N,N-di-(2-hydroxyethyl)-N,N-(fatty acid ester ethyl)ammonium chloride.

As a further optional constituent the agents used according to the invention can contain 0.01 to 10 wt. % of at least one polymer from the group of cationic and/or amphoteric polymers. Protection is or can be requested for polymer-containing agents for use according to the invention; polymers, in particular cationic polymers, contribute to the technical aim of the invention and hence to the solution of the technical object underlying the invention according to the application. Preferred polymers and the amounts in which they are contained in the compositions for use according to the invention are disclosed in the priority document DE 102009044972 on pages 18 to 27; the features cited therein clearly belong implicitly to the description of the invention contained in the submitted application and hence to the disclosure of said application.

A further preferred group of ingredients of the agents used according to the invention are vitamins, provitamins or vitamin precursors. These are described below:

The group of substances classed as vitamin A includes retinol (vitamin A₁) and 3,4-didehydroretinol (vitamin A₂). β-Carotene is the retinol provitamin. Suitable vitamin A components according to the invention are for example vitamin A acid and esters thereof, vitamin A aldehyde and vitamin A alcohol and esters thereof such as the palmitate and acetate. The agents used according to the invention contain the vitamin A component preferably in amounts from 0.05 to 1 wt. %, relative to the complete preparation.

The vitamin B group or the vitamin B complex includes inter alia vitamin B ₁ (thiamine), vitamin B₂ (riboflavin), vitamin B₃. The compounds nicotinic acid and nicotinic acid amide (niacinamide) are often included under this term. Preferred according to the invention is nicotinic acid amide, which is preferably contained in the agents used according to the invention in amounts from 0.05 to 1 wt. %, relative to the complete agent. It likewise includes vitamin B₅ (pantothenic acid, panthenol and pantolactone). Within the context of this group panthenol and/or pantolactone are preferably used. Derivatives of panthenol that can be used according to the invention are in particular the esters and ethers of panthenol as well as cationically derivatized panthenols. Individual representatives are for example panthenol triacetate, panthenol monoethyl ether and the monoacetate thereof as well as the cationic panthenol derivatives disclosed in WO 92/13829. The cited compounds of the vitamin B₅ type are preferably contained in the agents used according to the invention in amounts from 0.05 to 10 wt. %, relative to the complete agent. Amounts from 0.1 to 5 wt. % are particularly preferred. Vitamin B₆ (pyridoxine as well as pyridoxamine and pyridoxal) can also be used. Vitamin C (ascorbic acid). Vitamin C is used in the agents used according to the invention preferably in amounts from 0.1 to 3 wt. %, relative to the complete agent. Use in the form of the palmitic acid ester, glucosides or phosphates can be preferred. Use in combination with tocopherols can likewise be preferred. Vitamin E (tocopherols, in particular α-tocopherol). Tocopherol and derivatives thereof, which include in particular the esters such as acetate, nicotinate, phosphate and succinate, are preferably contained in the agents used according to the invention in amounts from 0.05 to 1 wt. %, relative to the complete agent. Vitamin F. The term “vitamin F” is conventionally understood to mean essential fatty acids, in particular linoleic acid, linolenic acid and arachidonic acid. Vitamin H. Vitamin H is the name given to the compound (3aS, 4S, 6aR)-2-oxohexahydrothienol[3,4-4-imidazole-4-valeric acid, although this is now more widely known by the trivial name biotin. Biotin is preferably contained in the agents used according to the invention in amounts from 0.0001 to 1.0 wt. %, in particular in amounts from 0.001 to 0.01 wt. %.

Hair treatment agents according to the invention are particularly preferred that as a care substance additionally contain, relative to their weight, 0.1 to 5 wt. %, preferably 0.2 to 4 wt. %, particularly preferably 0.25 to 3.5 wt. %, more preferably 0.5 to 3 wt. % and in particular 0.5 to 2.5 wt. % of vitamins and/or provitamins and/or vitamin precursors, which are preferably assigned to groups A, B, C, E, F and H, wherein preferred agents contain panthenol ((±)-2,4-dihydroxy-N-(3-hydroxypropyl)-3,3-dimethyl butyramide, provitamin B₅) and/or pantothenic acid (vitamin B₃, vitamin B₅) and/or niacin, niacinamide or nicotinamide (vitamin B₃) and/or L-ascorbic acid (vitamin C) and/or thiamine (vitamin B₁) and/or riboflavin (vitamin B₂, vitamin G) and/or biotin (vitamin B₇, vitamin H) and/or folic acid (vitamin B₉, vitamin B_{c} or vitamin M) and/or vitamin B₆ and/or vitamin B₁₂. Combinations of a 12 G homopolymer with tocopherols, in particular alpha-tocopherol, and combinations of a 20 G homopolymer with tocopherols, in particular alpha-tocopherol, are particularly preferred.

Particularly preferred hair treatment agents that are used according to the invention have the characterizing feature that as a care substance they contain, relative to their weight, 0.0001 to 1 wt. %, preferably 0.001 to 0.5 wt. % and particularly preferably 0.005 to 0.1 wt. % of at least one ubiquinone and/or at least one ubiquinol and/or at least one derivative of these substances, wherein agents that are particularly preferably to be used contain coenzyme Q10, preferably in an amount from 0.005 to 0.1 wt. %. As an alternative to or in addition to the particularly preferred ubiquinones, the agents used according to the invention can also contain plastoquinones (polyprenylated 2,3-dimethylbenzoquinone derivatives). Preferred agents used according to the invention have the characterizing feature that they contain 0.0002 to 4 wt. %, preferably 0.0005 to 3 wt. %, particularly preferably 0.001 to 2 wt. %, more preferably 0.0015 to 1 and in particular 0.002 to 0.5 wt. % of at least one plastoquinone. The prenyl side chain contains n prenyl units. Values for n from 1 to 20, preferably from 2 to 15 and in particular 5, 6, 7, 8, 9, 10 are preferred, wherein agents that are particularly preferably to be used contain a plastoquinone with n=9. A combination of at least one G homopolymer having 12 to 25 nucleotides with coenzyme Q10 is particularly preferred; preferred in particular is the combination of a 12 G homopolymer (guanine homopolymer having 12 nucleotides) with coenzyme Q10 or a 20 G homopolymer with coenzyme Q10.

As a further ingredient the agents used according to the invention can contain one or more amino acids to particular advantage. Amino acids that can particularly preferably be used according to the invention derive from the group comprising glycine, alanine, valine, leucine, isoleucine, phenylalanine, tyrosine, tryptophane, proline, aspartic acid, glutamic acid, asparagine, glutamine, serine, threonine, cysteine, methionine, lysine, arginine, histidine, β-alanine, 4-aminobutyric acid (GABA), betaine, L-cystine (L-cys), L-citrulline, L-theanine, 3′,4′-dihydroxy-L-phenylalanine (L-dopa), 5′-hydroxy-L-tryptophane, L-homocysteine, S-methyl-L-methionine, S-allyl-L-cysteine sulfoxide (L-alliin), L-trans-4-hydroxyproline, L-5-oxoproline (L-pyroglutamic acid), L-phosphoserine, creatine, 3-methyl-L-histidine, L-ornithine, wherein both the individual amino acids and mixtures can be used. Preferred agents used according to the invention contain one or more amino acids in narrower quantity ranges. Hair treatment agents that are preferred according to the invention have the characterizing feature that as a care substance they contain, relative to their weight, 0.01 to 5 wt. %, preferably 0.02 to 2.5 wt. %, particularly preferably 0.05 to 1.5 wt. %, more preferably 0.075 to 1 wt. % and in particular 0.1 to 0.25 wt. % of amino acid(s), preferably from the group comprising glycine and/or alanine and/or valine and/or lysine and/or leucine and/or threonine.

Particularly preferred are the following combinations of at least one G homopolymer having 12 to 22 nucleotides and a phosphodiester bond with glycine; at least one G homopolymer having 12 to 22 nucleotides and a phosphodiester bond with alanine; at least one G homopolymer having 12 to 22 nucleotides and a phosphodiester bond with valine; at least one G homopolymer having 12 to 22 nucleotides and a phosphodiester bond with lysine, at least one G homopolymer having 12 to 22 nucleotides and a phosphodiester bond with leucine, at least one G homopolymer having 12 to 22 nucleotides and a phosphodiester bond with threonine. Also particularly preferred are combinations of at least one G homopolymer having 12 to 22 nucleotides and a phosphorothioate linkage with glycine, at least one G homopolymer having 12 to 22 nucleotides and a phosphorothioate linkage with alanine, at least one G homopolymer having 12 to 22 nucleotides and a phosphorothioate linkage with valine, at least one G homopolymer having 12 to 22 nucleotides and a phosphorothioate linkage with lysine, at least one G homopolymer having 12 to 22 nucleotides and a phosphorothioate linkage with leucine, at least one G homopolymer having 12 to 22 nucleotides and a phosphorothioate linkage with threonine.

Particularly preferred is a combination of a 12 G homopolymer with glycine, a 12 G homopolymer with alanine, a 12 G homopolymer with valine, a 12 G homopolymer with lysine, a 12 G homopolymer with leucine, a 12 G homopolymer with threonine. Also particularly preferred are combinations of a 20 G homopolymer with glycine, a 20 G homopolymer with alanine, a 20 G homopolymer with valine, a 20 G homopolymer with lysine, a 20 G homopolymer with leucine, a 20 G homopolymer with threonine.

Agents that are preferred according to the invention contain as a care substance, relative to their weight, 0.01 to 15 wt. %, preferably 0.025 to 12.5 wt. %, particularly preferably 0.05 to 10 wt. %, more preferably 0.1 to 7.5 wt. % and in particular 0.5 to 5 wt. % of at least one 2-furanone derivative of the formula (Fur-I) and/or of the formula (Fur-II)

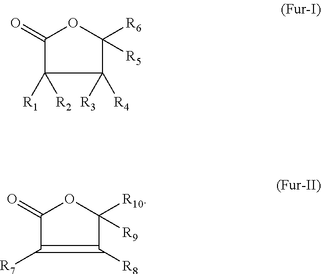

Protection is or can be requested for furanone derivative-containing agents for use according to the invention; 2-furanone derivatives contribute to the technical aim of the invention and hence to the solution of the technical object underlying the invention according to the application. Suitable 2-furanone derivatives and the amounts in which they are contained in the compositions for use according to the invention are disclosed in the priority document DE 102009044972 on pages 33 to 39; the features cited therein clearly belong implicitly to the description of the invention contained in the submitted application and hence to the disclosure of said application.

A further care substance that can preferably be used, which has activating properties, is taurine. Hair treatment agents that are preferred according to the invention contain as a care substance, relative to their weight, 0.01 to 15 wt. %, preferably 0,025 to 12.5 wt. %, particularly preferably 0.05 to 10 wt. %, more preferably 0.1 to 7.5 wt. % and in particular 0.5 to 5 wt. % of taurine (2-aminoethane sulfonic acid).

The agents used according to the invention can contain in addition to the optional further ingredients further substances which prevent, alleviate or cure hair loss. A content of active ingredients which stabilize the hair root is advantageous in particular. These substances are described below: Propecia (finasteride) is currently the only preparation that is approved worldwide and for which an effectiveness and tolerance has been proven in numerous studies. Propecia works by reducing the ability of DHT to form from testosterone. Minoxidil with or without supplementary additives is probably the oldest demonstrably effective hair growth agent. For the treatment of hair loss it should be used for external application only. There are hair lotions containing 2% to 5% minoxidil, also gels containing up to 15% minoxidil. The effectiveness increases with the dose, but in hair lotions minoxidil is soluble only in a content of up to 5%. In many countries hair lotions containing up to 2% minoxidil are available without a prescription. Spironolactone in the form of a hair lotion and in combination with minoxidil can be used for external application to combat hormonal influences on the hair follicles. Spironolactone works as an androgen receptor blocker, in other words binding of DHT to the hair follicles is prevented. In summary, cosmetic agents according to the invention are preferred which additionally contain, relative to their weight, 0.001 to 5 wt. % of hair root-stabilizing substances, in particular minoxidil and/or finasteride and/or ketoconazole.

A preferred form of formulation of the hair treatment agent according to the invention is in the form of hair tonics or hair lotions. These preferably contain at least one monohydric alcohol, at least one nucleic acid containing at least 55% guanine nucleotide, optionally a gelling agent and optionally at least one specific care enhancer.

The present invention also provides a hair treatment agent containing
(a) at least one nucleic acid containing at least 55% guanine nucleotide,
(b) 0.1 to 90 wt. % of at least one monohydric alcohol from the group comprising ethanol, n-propanol, isopropanol, n-butanol,
(c) 0 to 10 wt. % of at least one gelling agent.

All that has been stated in respect of the agents used according to the invention applies with necessary alterations to further preferred embodiments of the agents according to the invention.

Hair treatment agents containing guanine homopolymers having from 10 to 25 nucleotides are particularly preferred; the combination with 12 G homopolymer or 20 G homopolymer is preferred in particular.

The agents used according to the invention contain 0.1 to 90 wt. % of at least one monohydric alcohol from the group comprising ethanol, n-propanol, isopropanol, n-butanol, Of these, ethanol and/or isopropanol are particularly preferred. Particularly preferred hair treatment agents according to the invention have the characterizing feature that they contain, relative to their weight, 0.5 to 85 wt. %, preferably 1 to 80 wt. %, particularly preferably 5 to 75 wt. %, more preferably 10 to 70 wt. % and in particular 25 to 60 wt. % of ethanol and/or isopropanol.

Particularly preferred hair treatment agents contain exclusively ethanol. Hair treatment agents according to the invention that contain, relative to their weight, 5 to 80 wt. %, preferably 7.5 to 70 wt. %, particularly preferably 10 to 60 wt. %, more preferably 20 to 55 wt. % and in particular 25 to 50 wt. % of ethanol are particularly preferred here.

The agents used according to the invention can additionally contain a gelling agent. The adhesion of the agents to the hair can be improved and the application made more pleasant through the use of these gelling agents. Hair treatment agents according to the invention are preferred that, relative to their weight, contain 0.15 to 9 wt. %, preferably 0.2 to 8 wt. %, particularly preferably 0.25 to 7 wt. %, more preferably 0.3 to 6 wt. % and in particular 0.4 to 5 wt. % of at least one gelling agent from the groups of silicic acids and/or phyllosilicates and/or organophyllosilicates and/or metal soaps and/or hydrogenated castor oil and/or modified fat derivatives and/or polyamides and/or hydroxyethyl cellulose (HEC) and/or carboxymethyl cellulose (CMC) and/or hydroxypropyl methylcellulose (HPMC) and/or hydroxypropyl cellulose (HPC) and/or ethyl hydroxyethyl cellulose (EHEC) and/or polyvinyl alcohols and/or polyacrylic acid and/or polymethacrylic acids and salts thereof and/or polyacrylamides and/or polyvinyl pyrrolidone and/or polyethylene glycols and/or styrene-maleic anhydride copolymers and salts thereof and/or copolymers and/or terpolymers of acrylic acid and methacrylic acid and/or cellulose and/or starch and/or xanthan gum.

In a further preferred embodiment the agents used according to the invention, in particular also the hair lotions and/or hair tonics according to the invention, can contain emulsifiers (F).

In a further preferred embodiment the agents used according to the invention, in particular also the hair lotions and/or hair tonics according to the invention, can contain emulsifiers (F). Protection is or can be requested for emulsifier-containing agents for use according to the invention; emulsifiers contribute to the technical aim of the invention and hence to the solution of the technical object underlying the invention according to the application. Preferred emulsifiers and the amounts in which they are contained in the compositions according to the invention are disclosed in the priority document DE 102009044972 on pages 42 to 43; the features cited therein clearly belong implicitly to the description of the invention contained in the submitted application and hence to the disclosure of said application.

It has further proved advantageous if in addition to the polymer(s) from the group of cationic and/or amphoteric polymers further polymers (G) are contained in the agents used according to the invention. Protection is or can be requested for polymer-containing agents for use according to the invention; polymers contribute to the technical aim of the invention and hence to the solution of the technical object underlying the invention according to the application. Preferred polymers and the amounts in which they are contained in the compositions used according to the invention are disclosed in the priority document DE 102009044972 on pages 43 to 45; the features cited therein clearly belong implicitly to the description of the invention contained in the submitted application and hence to the disclosure of said application.

In a preferred embodiment of the invention an agent according to the invention can also contain UV filters (I). There are no general restrictions on the UV filters to be used according to the invention in terms of their structure and their physical properties. In fact all UV filters for use in the cosmetics sector whose absorption maximum is in the UVA (315-400 nm), UVB (280-315 nm) or UVC (<280 nm) range are suitable. UV filters having an absorption maximum in the UVB range, in particular in the range from approximately 280 to approximately 300 nm, are particularly preferred. The UV filters used according to the invention can be selected for example from substituted benzophenones, p-aminobenzoic acid esters, diphenyl acrylic acid esters, cinnamic acid esters, salicylic acid esters, benzimidazoles and o-aminobenzoic acid esters.

Examples of UV filters that can be used according to the invention are 4-aminobenzoic acid, N,N,N-trimethyl-4-(2-oxoborn-3-ylidene methyl)aniline methyl sulfate, 3,3,5-trimethyl cyclohexyl salicylate (Homosalate), 2-hydroxy-4-methoxybenzophenone (Benzophenone-3; Uvinul®M 40, Uvasorb®MET, Neo Heliopan®BB, Eusolex®4360), 2-phenylbenzimidazole-5-sulfonic acid and potassium, sodium and triethanolamine salts thereof (Phenylbenzimidazole sulfonic acid; Parsol®HS; Neo Heliopan®Hydro), 3,3′-(1,4-phenylenedimethylene)-bis(7,7-dimethyl-2-oxobicyclo-[2.2.1]hept-1-yl-methanesulfonic acid) and salts thereof, 1-(4-tert-butylphenyl)-3-(4-methoxyphenyl)propane-1,3-dione (Butyl methoxydibenzoylmethane; Parsol®1789, Eusolex®9020), α-(2-oxoborn-3-ylidene)toluene-4-sulfonic acid and salts thereof, ethoxylated 4-aminobenzoic acid ethyl ester (PEG-25 PABA; Uvinul®P 25), 4-dimethylaminobenzoic acid-2-ethylhexyl ester (Octyl Dimethyl PABA; Uvasorb®DMO, Escalol®507, Eusolex®6007), salicylic acid-2-ethylhexyl ester (Octyl Salicylate; Escalol®587, Neo Heliopan®OS, Uvinul®018), 4-methoxycinnamic acid isopentyl ester (Isoamyl p-Methoxycinnamate; Neo Heliopan®E 1000), 4-methoxycinnamic acid-2-ethylhexyl ester (Octyl Methoxycinnamate; Parsol®MCX, Escalol®557, Neo Heliopan®AV), 2-hydroxy-4-methoxybenzophenone-5-sulfonic acid and the sodium salt thereof (Benzophenone-4; Uvinul®MS 40; Uvasorb®S 5), 3-(4′-methylbenzylidene)-D,L-camphor (4-Methylbenzylidene camphor; Parsol©5000, Eusolex®6300), 3-benzylidene camphor (3-Benzylidene camphor), 4-isopropylbenzyl salicylate, 2,4,6-trianilino-(p-carbo-2′-ethylhexyl-1′-oxi)-1,3,5-triazine, 3-imidazol-4-yl acrylic acid and ethyl esters thereof, polymers of N-{(2 and 4)-[2-oxoborn-3-ylidene methyl]benzyl}acrylamide, 2,4-dihydroxybenzophenone (Benzophenone-1; Uvasorb®20 H, Uvinul®400), 1,1′-diphenylacrylonitrilic acid-2-ethylhexyl ester (Octocrylene; Eusolex®OCR, Neo Heliopan®Type 303, Uvinul®N 539 SG), o-aminobenzoic acid menthyl ester (Menthyl Anthranilate; Neo Heliopan®MA), 2,2′,4,4′-tetrahydroxybenzophenone (Benzophenone-2; Uvinul®D-50), 2,2′-dihydroxy-4,4′-dimethoxybenzophenone (Benzophenone-6), 2,2′-dihydroxy-4,4′-dimethoxybenzophenone-5-sodium sulfonate and 2-cyano-3,3-diphenylacrylic acid-2′-ethylhexyl ester. 4-Aminobenzoic acid, N,N,N-trimethyl-4-(2-oxoborn-3-ylidene methyl)aniline methyl sulfate, 3,3,5-trimethyl cyclohexyl salicylate, 2-hydroxy-4-methoxybenzophenone, 2-phenylbenzimidazole-5-sulfonic acid and potassium, sodium and triethanolamine salts thereof, 3,3′-(1,4-phenylenedimethylene)-bis(7,7-dimethyl-2-oxobicyclo-[2.2.1]hept-1-yl-methanesulfonic acid) and salts thereof, 1-(4-tert-butylphenyl)-3-(4-methoxyphenyl)propane-1,3-dione, α-(2-oxoborn-3-ylidene)toluene-4-sulfonic acid and salts thereof, ethoxylated 4-aminobenzoic acid ethyl ester, 4-dimethylaminobenzoic acid-2-ethylhexyl ester, salicylic acid-2-ethylhexyl ester, 4-methoxycinnamic acid isopentyl ester, 4-methoxycinnamic acid-2-ethylhexyl ester, 2-hydroxy-4-methoxybenzophenone-5-sulfonic acid and the sodium salt thereof, 3-(4′-methylbenzylidene)-D,L-camphor, 3-benzylidene camphor, 4-isopropylbenzyl salicylate, 2,4,6-trianilino-(p-carbo-2′-ethylhexyl-1′-oxi)-1,3,5-triazine, 3-imidazol-4-yl acrylic acid and ethyl esters thereof, polymers of N-{(2 and 4)-[2-oxoborn-3-ylidene methyl]benzyl}acrylamide are preferred. Most particularly preferred according to the invention are 2-hydroxy-4-methoxybenzophenone, 2-phenylbenzimidazole-5-sulfonic acid and potassium, sodium and triethanolamine salts thereof, 1-(4-tert-butylphenyl)-3-(4-methoxyphenyl)propane-1,3-dione, 4-methoxycinnamic acid-2-ethylhexyl ester and 3-(4′-methylbenzylidene)-D,L-camphor.

UV filters whose molar extinction coefficient at the absorption maximum is above 15,000, in particular above 20,000, are preferred.

It has moreover been found that with structurally similar UV filters, the non-water-soluble compound has in many cases the greater effect in the context of the teaching according to the invention as compared with water-soluble compounds that differ therefrom by one or more additional ionic groups. Within the context of the invention non-water-soluble is understood to mean UV filters that dissolve in water at 20° C. by no more than 1 wt. %, in particular no more than 0.1 wt. %. These compounds should furthermore be soluble in conventional cosmetic oil components at room temperature by at least 0.1, in particular at least 1 wt. %. The use of non-water-soluble UV filters can therefore be preferred according to the invention.

According to a further embodiment of the invention UV filters having a cationic group, in particular a quaternary ammonium group, are preferred. These UV filters have the general structure U-Q.

The structural part U denotes a group that absorbs UV radiation. This group can in principle be derived from the aforementioned known UV filters that are suitable for use in the cosmetic sector by substituting a group, generally a hydrogen atom, of the UV filter with a cationic group Q, in particular having a quaternary amino function.

Compounds that can be derived from the structural part U are for example substituted benzophenones, p-aminobenzoic acid esters, diphenyl acrylic acid esters, cinnamic acid esters, salicylic acid esters, benzimidazoles and o-aminobenzoic acid esters.

Structural parts U that derive from cinnamic acid amide or from N,N-dimethylaminobenzoic acid amide are preferred according to the invention. The structural parts U can in principle be chosen such that the absorption maximum of the UV filters can lie in both the UVA range (315-400 nm) and in the UVB range (280-315 nm) or the UVC range (<280 nm). UV filters having an absorption maximum in the UVB range, in particular in the range from approximately 280 to approximately 300 nm, are particularly preferred.

Depending also on the structural part Q, the structural part U is furthermore preferably chosen such that the molar extinction coefficient of the UV filter at the absorption maximum is above 15,000, in particular above 20,000.

The structural part Q preferably contains a quaternary ammonium group as the cationic group. This quaternary ammonium group can in principle be linked directly to the structural part U, such that the structural part U is one of the four substituents of the positively charged nitrogen atom. However, one of the four substituents at the positively charged nitrogen atom is preferably a group, in particular an alkylene group having 2 to 6 carbon atoms, that functions as a link between the structural part U and the positively charged nitrogen atom.

The group Q advantageously has the general structure —(CH₂)ₓ—N⁺R¹R²R³X⁻, in which x denotes a whole number from 1 to 4, R¹ and R² independently of each other denote C₁₋₄ alkyl groups, R³ denotes a C₁₋₂₂ alkyl group or a benzyl group and X⁻ denotes a physiologically tolerable anion. In the context of this general structure x preferably denotes the number 3, R¹ and R² each denote a methyl group and R³ denotes either a methyl group or a saturated or unsaturated, linear or branched hydrocarbon chain having 8 to 22, in particular 10 to 18, carbon atoms.

Physiologically tolerable anions are for example inorganic anions such as halides, in particular chloride, bromide and fluoride, sulfate ions and phosphate ions as well as organic anions such as lactate, citrate, acetate, tartrate, methosulfate and tosylate.

Two preferred UV filters having cationic groups are the compounds cinnamic acid amidopropyl trimethylammonium chloride (lncroquat®UV-283) and dodecyl dimethylaminobenzamidopropyl dimethylammonium tosylate (Escalol® HP 610), which are available as commercial products.

The teaching according to the invention naturally also encompasses the use of a combination of a plurality of UV filters. In the context of this embodiment the combination of at least one non-water-soluble UV filter with at least one UV filter having a cationic group is preferred. The UV filters (I) are conventionally contained in the agents used according to the invention in amounts from 0.1 to 5 wt. %, relative to the complete agent. Amounts from 0.4 to 2.5 wt. % are preferred. In the agents used according to the invention the UV filters improve the results of the repigmentation process, in the long term in particular, and are therefore particularly suitable. The aforementioned UV filters are particularly preferably combined with at least one 12 G homopolymer or 20 G homopolymer.

The agents used according to the invention can moreover contain a 2-pyrrolidinone-5-carboxylic acid and derivatives thereof (J). The sodium, potassium, calcium, magnesium or ammonium salts are preferred, in which the ammonium ion bears one to three C₁ to C₄ alkyl groups in addition to hydrogen. The sodium salt is most particularly preferred. The amounts used in the agents used according to the invention are preferably 0.05 to 10 wt. %, relative to the complete agent, particularly preferably 0.1 to 5, and in particular 0.1 to 3 wt. %.

Finally the agents used according to the invention can also contain plant extracts (L). These extracts are conventionally produced by extraction of the entire plant. It can also be preferable in individual cases, however, to produce the extracts exclusively from flowers and/or leaves of the plant.

Regarding the plant extracts that can be used according to the invention, reference is made in particular to the extracts listed in the table beginning on page 44 of the 3^{rd} edition of the Leitfaden zur Inhaltsstoffdeklaration kosmetischer Mittel, published by the Industrieverband Körperpflege- and Waschmittel e.V. (IKW), Frankfurt.

The extracts from green tea, oak bark, stinging nettle, witch hazel, hops, henna, chamomile, burdock, horsetail, whitethorn, lime blossom, almond, aloe vera, pine, horse chestnut, sandalwood, juniper, coconut, mango, apricot, lemon, wheat, kiwi, melon, orange, grapefruit, sage, rosemary, birch, mallow, lady's smock, wild thyme, yarrow, thyme, melissa, restharrow, coltsfoot, marshmallow, meristem, ginseng and ginger root are preferred above all according to the invention.

The extracts from green tea, oak bark, stinging nettle, witch hazel, hops, chamomile, burdock, horsetail, lime blossom, almond, aloe vera, coconut, mango, apricot, lemon, wheat, kiwi, melon, orange, grapefruit, sage, rosemary, birch, lady's smock, wild thyme, yarrow, restharrow, meristem, ginseng and ginger root are particularly preferred.

The extracts from green tea, almond, aloe vera, coconut, mango, apricot, lemon, wheat, kiwi and melon are most particularly suitable for the use according to the invention.

Water, alcohols and mixtures thereof can be used as extracting agents to produce the cited plant extracts. Of the alcohols, low alcohols such as ethanol and isopropanol, but in particular polyhydric alcohols such as ethylene glycol and propylene glycol, are preferred, both as the sole extracting agent and mixed with water. Plant extracts based on water/propylene glycol in the ratio 1:10 to 10:1 have proved to be particularly suitable.

The plant extracts can be used according to the invention in both pure and diluted form. If they are used in diluted form they conventionally contain approximately 2 to 80 wt. % of active substance and as the solvent the extracting agent or mixture of extracting agents used to obtain them.

It can furthermore be preferable to use mixtures of a plurality of different plant extracts, in particular two, in the agents used according to the invention.

It can additionally prove advantageous if penetration auxiliaries and/or swelling agents (M) are contained in the agents used according to the invention. They include for example urea and urea derivatives, guanidine and derivatives thereof, arginine and derivatives thereof, water glass, imidazole and derivatives thereof, histidine and derivatives thereof, benzyl alcohol, glycerol, glycol and glycol ethers, propylene glycol and propylene glycol ethers, for example propylene glycol monoethyl ether, carbonates, hydrogen carbonates, diols and triols, and in particular 1,2-diols and 1,3-diols such as for example 1,2-propanediol, 1,2-pentanediol, 1,2-hexanediol, 1,2-dodecanediol, 1,3-propanediol, 1,6-hexanediol, 1,5-pentanediol, 1,4-butanediol.

All that has been stated in respect of the agents used according to the invention applies with necessary alterations to further preferred embodiments of the use according to the invention.

The present invention also provides a method for influencing the natural pigmentation process of skin and/or skin appendages, in particular for stimulating the natural pigmentation process, in particular melanogenesis and/or pigmentation of the hair, for preventing and/or reducing graying of the hair and/or for repigmenting gray hair, wherein at least one nucleic acid containing at least 55% guanine nucleotide is brought into contact topically with hair and/or skin.

All that has been stated in respect of the uses and agents according to the invention applies with necessary alterations to further preferred embodiments of the method according to the invention.

### EXAMPLES

### Example 1

### Proof of the Differential Expression of Melanogenesis-Relevant Genes

The ligands involved in melanogenesis, such as SCF or alpha-MSH (melanocyte stimulating hormone alpha) bind to different receptors, through which the corresponding signal is transmitted into the cell's interior. The receptor for SCF is ckit, the receptor for alpha-MSH is MCR-1 (melanocortin receptor 1). Substances that bring about a change in the expression of MCR-1 and/or ckit can influence melanogenesis. If an induction (up-regulation or stimulation) of the gene expression of the corresponding receptors occurs, melanogenesis is assumed to be stimulated.

Gp100 is a protein that occurs in the membrane of melanosomes and stabilizes them. Since more melanin is produced in the cells following application of substances that positively influence melanogenesis, an increase in the melanosomes necessary for transport also occurs. A substance that induces the gene expression of gp100 is therefore a pigmentation-stimulating active ingredient.

Particularly preferred substances that stimulate the natural pigmentation process of skin and/or skin appendages, in particular hair or hair follicles, are those that both bring about the gene expression of MCR-1 and/or ckit and induce the gene expression of gp100.

Determining the extent of the change in gene expression following an application of such substances to suitable cells/cell systems/tissue cultures can provide evidence of the effectiveness of the active ingredient.

Differential gene expression was determined by means of quantitative RT-PCR. After preparing three-dimensional organotypical hair follicle cell cultures from dermal papilla cells on microcarriers, they were incubated for 48 h with a nucleic acid consisting of 20 guanine nucleotides, linked via phosphorothioate linkages. In order to perform PCR the RNA was first isolated from the organotypical cell cultures with the aid of an RNeasy Mini Kit from Qiagen and transcribed into cDNA by reverse transcription. In the subsequent PCR reaction, which is performed for each gene with the aid of gene-specific primers and which serves to amplify the required gene sections, the formation of PCR products is detected online via a fluorescence signal. The fluorescence signal is proportional to the amount of PCR product formed. The stronger the expression of a particular gene, the greater the amount of PCR product formed and the higher the fluorescence signal.

To quantify the gene expression the untreated control is set to 1 and the expression of the gene to be determined is referenced thereto (x-times expression). Values greater than or equal to the 1.8 times expression or less than or equal to the 0.5 times expression of the untreated control are classed as being expressed in a significantly differential manner. Values greater than or equal to the 1.5 times expression or less than or equal to the 0.7 times expression of the untreated control are classed as being expressed in a tendentially differential manner.

TABLE 1
Influence of 20G-PTO on the expression of
melanogenesis-regulating genes

| | | MCR1 | | gp100 | | ckit | |
|---|---|---|---|---|---|---|---|
| | Cone [μM] | Mean | SD | Mean | SD | Mean | SD |
| Untreated | | 1.00 | 0.26 | 1.00 | 0.13 | 1.00 | 0.16 |
| 20G PTO | 4 | 11.74 | 1.66 | 20.14 | 5.86 | 9.00 | 0.84 |

For 20G-PTO in a usage concentration of 4 μM a significant induction in comparison to the untreated control was demonstrated for all three investigated genes.

### Example 2

### Proof of the Induction of gp100 by Western Blot Analysis

In addition to the induction of gene expression, translation into the corresponding proteins is likewise of importance for the influencing of cellular mechanisms. The expression of gp100 was therefore additionally demonstrated at a protein level by western blot technology. To this end the proteins were extracted from three-dimensional organotypical hair follicle cell cultures prepared from dermal papilla cells and hair follicle melanocytes, which had been treated for 72 h with a 20 G homopolymer linked via phosphorothioate bonds (PTO), and separated by electrophoresis. The proteins were then transferred to a nitrocellulose membrane (blotting). The desired protein can then be detected on this membrane by means of specific antibodies and quantified in relation to the untreated control (=1).

TABLE 2
Influence of 20G-PTO on the expression of
gp100 relative to the untreated control

| | Conc [μM] | gp100 |
|---|---|---|
| Untreated | | 1.00 |
| 20G PTO | 4 | 3.34 |

With a usage concentration of 4 μM 20G-PTO, the 20 G homopolymer with phosphorothioate linkage, the protein expression of gp100 was able to be induced in comparison to the untreated control.

### Example 3

### Stimulation of Melanin Synthesis

Melanin is a dye that is produced and stored in the melanosomes of the melanocytes. Melanin gives the hair its intrinsic color, the coloration being formed by a mixture of two types of melanin, eumelanin and pheomelanin. Melanogenesis is a complicated and highly regulated synthesis process. Tyrosine is converted first by the enzyme tyrosinase into L-dihydroxyphenylalanine (L-DOPA) and then via a plurality of intermediate steps into the various melanin pigments. An active ingredient that positively influences melanogenesis and leads to an increased melanin content in the hair follicle melanocytes is particularly suitable for influencing the natural pigmentation process of skin and/or skin appendages, preventing hair graying and/or stimulating pigmentation.

In order to assess the melanin content, isolated, organ-cultivated hair follicles were treated for 7 days with a 12G nucleic acid, a nucleic acid having a succession of 12 guanine nucleotide units, linked via phosphodiester bonds. Untreated hair follicles served as a control. After 7 days the hair follicles were fixed to produce histological sections and 10 μm sections were prepared with the aid of a cryomicrotome. Melanin staining was then carried out using the Fontana-Masson protocol. The sections stained in this way were evaluated by imaging with the aid of the ImageJ program of the National Institute of Health and the melanin content in the treated follicles was compared with the untreated examples.

TABLE 3
Melanin content in isolated hair follicles
after treatment with a 12G nucleic acid

| | |
|---|---|
| | Melanin content [%] |
| Conc | d 7 |

| | [μM] | Mean | SD |
|---|---|---|---|
| Untreated | | 100 | 4.6 |
| 12G | 10 | 110 | 5.7 |

With a usage concentration of 10 μM of the 12 G homopolymer the melanin content of the cultivated hair follicles was able to be slightly induced in comparison to the untreated control.

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

What is claimed is:

1. A method for influencing the natural pigmentation process of skin or appendages thereof, comprising:
topically contacting the skin or appendages thereof with at least one nucleic acid containing at least 55% guanine nucleotide.

2. The method according to claim 1, wherein the step of topcially contacting the skin or appendages thereof with at least one nucleic acid stimulates at least one sub-step of the natural pigmentation process.

3. The method according to claim 1, wherein the step of topcially contacting the skin or appendages thereof with at least one nucleic acid stimulates the natural pigmentation process of the hair.

4. The method according to claim 3, wherein the step of topcially contacting the skin or appendages thereof with at least one nucleic acid improves the pigmentation of the hair.

5. The method according to claim 1, wherein the step of topcially contacting the skin or appendages thereof with at least one nucleic acid influences melanogenesis in the hair follicle.

6. The method according to claim 1, wherein the step of topcially contacting the skin or appendages thereof with at least one nucleic acid reduces graying of the hair.

7. The method according to claim 1, wherein the step of topcially contacting the skin or appendages thereof with at least one nucleic acid repigments gray hair.

8. The method according to claim 1, wherein the at least one nucleic acid has a chain length of from 4 to 100 nucleotides.

9. The method according to claim 8, wherein the at least one nucleic acid has a chain length of from 8 to 30 nucleotides.

10. The method according to claim 8, wherein the at least one nucleic acid has a chain length of 10 to 25 nucleotides.

11. The method according to claim 1, wherein the at least one nucleic acid contains at least 65% guanine nucleotide in the nucleic acid sequence.

12. The method according to claim 11, wherein the nucleic acid is a G homopolymer.

13. The method according to claim 1, wherein the nucleic acid has a sequence of at least 4 guanine nucleotides.

14. The method according to claim 1, wherein the nucleic acid is completely or partially chemically modified.

15. The method according to claim 14, wherein the nucleic acid has at least one phosphorothioate linkage.

16. The method according to claim 1, wherein the step of topcially contacting the skin or appendages thereof with at least one nucleic acid comprises applying to the skin or appendages thereof a cosmetic agent that contains the at least one nucleic acid in a total amount from 0.0000001 to 5 wt. % relative to the total weight of the agent.

17. A hair treatment agent containing
a. at least one nucleic acid containing at least 55% guanine nucleotide;
b. 0.1 to 90 wt. % of at least one monohydric alcohol from the group comprising ethanol, n-propanol, isopropanol, n-butanol; and
c. 0 to 10 wt. % of at least one gelling agent.
